(19) United States
(12) Patent Application Publication
(10) Pub. No.: US 2002/0043051 A1
(43) Pub. Date: Apr. 18, 2002

(54) Container and method of sealing

(75) Inventors: Keith Manica, Lakewood, CO (US); Dennis J. Hlavinka, Golden, CO (US); Frank Corbin III, Littleton, CO (US)

Correspondence Address: Gambro, Inc., 10810 W. Collins Ave., Lakewood, CO, 80215, US

(73) Assignee: Gambro, Inc

(21) Appl. No.: 09/982,298

(22) Filed: Oct. 16, 2001

Related U.S. Application Data
(60) Provisional application No. 60/241,091, filed on Oct. 17, 2000.
(60) Provisional application No. 60/312,436, filed on Aug. 15, 2001.

(51) Int. Cl. B65B 3/04

(52) U.S. Cl. 53/469

(57) ABSTRACT

A method and apparatus for use in inactivating pathogens in fluids such as blood or blood components. The apparatus includes a container having a main body portion, a plurality of openings into the main body portion of the container, and at least one weld which partially surrounds at least one opening into the main body portion of the container. The method includes flowing fluid into the container via at least one of the plurality of openings and sealing at least one opening closed by extending the partially surrounding weld so that it completely surrounds the opening.

### CROSS-REFERENCES TO RELATED APPLICATIONS

This Application claims priority from U.S. application Ser. No. 60/241091 filed Oct. 17, 2000 and from U.S. application Ser. No. 60/312436 filed Aug. 15, 2001 both of which are incorporated herein in their entirety to the extent not incompatible herewith.

### FIELD OF THE INVENTION

The present invention generally relates to the collection and storage of blood or blood components. More specifically, the present invention relates to a container which can be used in the inactivation of pathogens in blood and blood components, which is adapted to be sealed so as to create a sealing weld across the fluid inlet after filling the container.

### BACKGROUND OF THE INVENTION

Contamination of human blood and blood components with pathogens such as human immunodeficiency virus (HIV), hepatitis and bacteria create a serious risk for patients who receive blood or blood components via blood transfusions. Whole blood, packed red cells, platelets and plasma (either fresh or fresh frozen) are examples of such transfusable blood and blood components. To help combat this problem, blood and fluids can be decontaminated using pathogen inactivating agents or photosensitizers which, when activated thereby, inactivate pathogens contained in the blood or fluid but do not destroy the biological activity of the blood or fluid components.

The pathogen inactivation agents which may be useful in this invention include the class of photosensitizers known in the art to be useful for inactivating microorganisms. A “photosensitizer” is defined as any compound which absorbs radiation of one or more defined wavelengths and subsequently transfers the absorbed energy to an energy acceptor. Thus, such photosensitizers may be activated by the application of electromagnetic spectra (e.g., light) to then inactivate certain pathogens with which they may interact. Non-photosensitized pathogen inactivation agents are also considered within the realm of the present invention.

Various photosensitizers have been proposed for use as blood or blood component additives to inactivate pathogens in body fluids. Examples of non-endogenous photosensitizers that have been proposed for use as blood or blood component additives include porphyrins, psoralens, acridines, toluidines, flavins (acriflavin hydrochloride), phenothiazine derivatives, coumarins, quinolines, quinones, anthroquinones and dyes such as neutral red and methylene blue. Other molecules such as chlorpromazine, protoporphyrin and ethyleneimine have been suggested for use as blood additives or blood component additives.

Other categories of photosensitizers are endogenous pathogen inactivation agents, such as 7,8,10-trimethylisoalloxazine (lumiflavin), 7,8-dimethylalloxazine (lumichrome), isoalloxazine-adenine dinucleotide (flavin adenine dinucleotide [FAD]), alloxazine mononucleotide (flavin mononucleotide [FMN] and riboflavin-5-phosphate), vitamin K and vitamin L and their metabolites and precursors, napththoquinones, naphthalenes and naphthols as well as their derivatives. One preferred example of an endogenous photosensitizer contemplated for use with this invention is an alloxazine such as 7,8-dimethyl-10-ribityl isoalloxazine, commonly known as riboflavin. An advantage of using endogenous photosensitizer to inactivate blood contaminants is that endogenous photosensitizers are not inherently toxic to the blood cells and if photoactivated do not yield toxic photoproducts after photoradiation. Therefore, no removal or purification step is required after the decontamination process, and the treated product then can be stored or returned directly to a patient's body.

One method of decontaminating blood or blood components includes mixing an effective amount of a photosensitizer with the fluid to be decontaminated; then exposing the fluid to an amount of photoradiation at an appropriate wavelength sufficient to activate the photosensitizer, and allowing the activated agent to interfere with the pathogens contained within the fluid such that the pathogens contained in the fluid are inactivated. In photoinactivation examples, the wavelength of light used will depend on the photosensitizing agent selected. Preferably the light source is a fluorescent or luminescent source providing light of about 300 nm to about 700 nm, and more preferably about 340 nm to 650 nm. The light source or sources may provide light in the visible range, ultraviolet range, or a mixture of light in both the visible and ultraviolet ranges.

In some photosensitizer methods, the material to be decontaminated is flowed through an entry port into a photopermeable bag or like container. The term “photopermeable” means that the material of the container is adequately transparent to photoradiation. The photosensitizer agent may be in a dry form such as powder, pill or a tablet. The photopermeable container may contain the photosensitizer, or the agent may be added to the fluid before its introduction into the container, or the photosensitizer may be dissolved in a solution which is then added to the fluid which is to undergo pathogen inactivation. In any of these cases, the container is preferably rocked on a shaker table or the like to mix the photosensitizers with the fluid. In the preferred embodiment using a pathogen inactivation agent such as a photosensitizer; such mixing preferably occurs while being exposed to the photoradiation source and the rocking preferably provides mixing to distribute the agent throughout the fluid to be pathogen inactivated. The pathogen inactivated fluid may then flow out of the inactivation container into a storage container through an exit port, or may be kept in the inactivation container until transfused.

Polymeric bags and other similar containers are useful as photopermeable bags are typically constructed from flexible sheets of a polymeric material welded together by welds or seals along the outer border zones of the container. Such containers may alternatively be blow molded. If the polymeric container is blow molded, the container would not likely have welds or seals along the outer border zones of the container.

It is known in the art to seal off the inlets and/or outlets of a blood component container during use, such as is done with sample bags and the like, by using electromagnetic or radio frequency (RF) energy as shown in U.S. Pat. No. 5,685,875 to Hlavinka. Various sorts of electromagnetic energies may be applied to create such seals, however, the choice of materials used in the inlet and/or outlet ports is related to the chosen sealing method. For example, if RF energy is to be used, the inlet and outlet ports can be constructed from material that is excitable in response to RF energy. It is well known that various thermoplastic materials such as polyvinyl chloride (PVC), having high dielectric loss coefficients, may be melted by the application of RF electromagnetic fields, which excite and dielectrically heat the thermoplastic materials. U.S. Pat. No. 4,013,860, issued to Hosterman, et al for a “Hand-Held Electro-Mechanism Sealer” describes one example of an RF sealer which can be hand-held, and can be used to seal PVC or other like polymeric containers or tubes. In one embodiment, the sealer may compress the plastic material between two jaws. RF energy may then be applied to the jaws, creating an electromagnetic field in the RF spectrum. The electromagnetic field excites and dielectrically heats the plastic material held between the jaws, partially melting the plastic material. The partially melted material welds together to form a hermetic fluid-tight seal.

Although PVC is commonly used in applications involving heat sealable material, PVC is not as permeable to photoradiation as other available polymeric materials. Furthermore, PVC may become brittle when frozen, which is undesirable as various blood components such as plasma may be frozen in storage either before or after being inactivated. Another type of polymeric material used to make containers that are capable of being frozen and thawed, as well as being highly photopermeable, includes the class of polyolefin materials. Polyolefin materials are not, however, generally excitable in response to RF energy, and hence are not as simply sealed or welded together using RF energy.

A fluid container such as that used in the present invention may contain a number of ports which provide access to the interior of the container. Such ports are typically manufactured, at least in part, out of polymeric materials which are usually more rigid than the main body portion of the container. During viral or pathogen inactivation a portion of the fluid to be inactivated may become trapped or remain within one or more of these ports, such that the photosensitizer is not able to be adequately distributed or mixed with the fluid to be inactivated. Another problem with such ports is that they may be substantially opaque, which may prevent the passage of photoradiation to the fluid contained within the port. Fluid thus trapped within the ports may still contain pathogen contaminants after the inactivation process is completed, and such contaminants may then redistribute within the otherwise inactivated fluid, reinfecting the fluid.

### BRIEF SUMMARY OF THE INVENTION

The present invention relates to a method and apparatus for inactivating pathogens in a fluid containing pathogens, wherein the fluid is contained within a preferably polymeric photopermeable container; the method preferably comprises the steps of flowing a fluid to be inactivated through a first port into the main body portion of the container, sealing the first port closed in order to isolate the port from the main interior body portion of the container, and exposing the fluid to light to activate a photosensitizer contained therein. A photosensitizer such as riboflavin which is photoactivated when exposed to light of an appropriate wavelength may be contained in the fluid as it enters the port, or may be mixed with the fluid in the container. Examples of preferred polymer photopermeable containers that may be used include, but are not limited to, a cuvette, a bottle, a chamber, a vessel, a tube, a beaker, a flask and/or a bag. In the preferred embodiments of this invention, it is envisioned that a bag would be used.

Another aspect of the present invention is to provide a polymeric photopermeable container for containing fluid to be inactivated that includes a main body portion to contain the fluid with a tube for passing fluid into the main body portion. The bag has a seal or weld partially surrounding the tube for passing fluid into the main body portion of the bag. The bag also includes a frangible connector for passing fluid out of the bag after the fluid has been inactivated.

Another aspect of the present invention includes a method of sealing the tube for passing fluid into the bag off from the main body portion of the bag using a hand held sealer.

### BRIEF DESCRIPTION OF THE DRAWINGS

FIG. 1 shows a top view of a polymeric container according to the present invention.

FIG. 2 shows a partially broken away cross-sectional view of the open jaws of a hand-held heat sealer before sealing closed the entry port of the polymeric container of FIG. 1.

FIG. 3 shows a partially broken away cross-sectional view of the closed jaws of a hand-held heat sealer sealing the entry port of the polymeric container of FIG. 1.

FIG. 4 shows a partially broken away cross-sectional view of the open jaws of a hand-held heat sealer after the entry port on the polymeric container of FIG. 1 has been sealed.

FIG. 5 shows a partially broken away cross-sectional view of the open jaws of a hand-held sealer placed over the alignment hole in preparation for sealing closed the entry port on the polymeric container of FIG. 1.

FIG. 6 shows a top view of a container such as that shown in FIG. 1 after the entry port is sealed in a continuous seal in alignment with the inside edge of the pre-formed manufactured seal.

FIG. 7 shows a top view of an alternative embodiment of a container wherein a piece of RF excitable material is placed over the location to be sealed.

FIG. 8 shows a partially broken away sectional view of the open jaws of a hand-held heat sealer with RF excitable selectively engageable sleeves before sealing the entry port closed on the container.

FIG. 9 shows a top view of a container containing fluid and a photosensitizer on a shaker table to agitate the fluid while exposing the fluid to photoradiation from a light source.

FIG. 10 shows a top view of an alternative polymeric container according to the present invention.

FIG. 11 shows another alternative embodiment of the present invention.

FIG. 12 shows another alternative embodiment of the present invention.

FIG. 13 shows the alternative embodiment of FIG. 10 with the tubing sealed off from the main body portion of the bag.

FIG. 14 shows the alternative embodiment of FIG. 11 with the tubing sealed off from the main body portion of the bag.

FIG. 15 shows the alternative embodiment of FIG. 12 with the tubing sealed off from the main body portion of the bag.

FIG. 16 shows a partially broken away plan view of an alternative port which may be used with the present invention.

### DESCRIPTION OF THE PREFERRED EMBODIMENTS

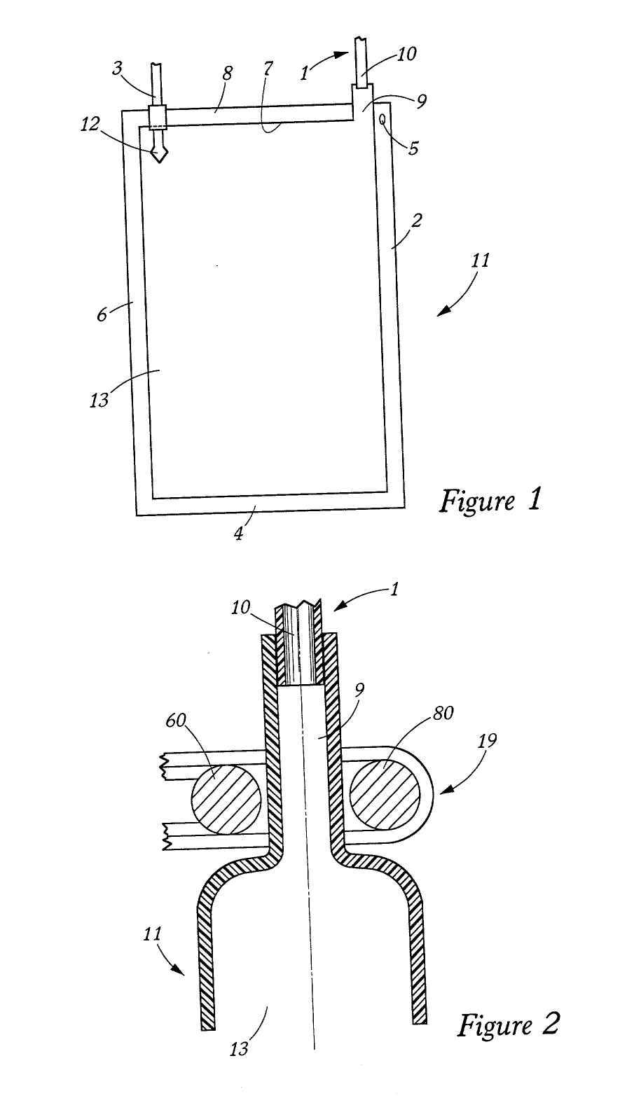

FIG. 1 shows a top plan view of one type of polymeric container or bag 11 which may be used in accordance with the present invention for inactivating any pathogens or viruses in fluids. The container or bag 11 is preferably a polymeric type which is sealed or welded around its outer border zones during manufacture in a continuous manner to form pre-formed seals or welds 2, 4, 6, 8 respectively, around all sides of the container 11. Continuous here refers to the hermetic fluid-tight seal around the entire periphery of bag 11, except for openings provided for inlet or outlet ports such as ports 1 and 3. The continuous pre-formed seals or welds create an interior space or main body portion 13. Two ports or openings 1 and 3 allow fluid ingress and egress; that is, the first port 1 is also entry or inlet port 1 which provides fluid ingress, port 3 is also the second exit or outlet port 3 which provides fluid egress. In FIG. 1, the container 11 is shown with entry port 1 open to allow the fluid which is to be pathogen inactivated to flow into the interior space 13 of the container 11. In the preferred embodiment described herein, the container 11 has two ports 1 and 3; however, the container could have any number of ports as required for its particular purpose, and still remain within the spirit and scope of the present invention.

Entry port 1 preferably includes two portions, a relatively rigid first portion 10 and a flexible second portion 9. Relatively rigid merely means more rigid than the flexible portion, but is not intended to disallow the use of substantially flexible tubes such as are commonly in use with blood component containers. The relatively rigid portion may be due to a thicker cross-section of tubing as compared to the cross-section of the adjacent film of the container. The relatively rigid first portion 10 extends from the exterior of the container toward the interior, or main body portion 13 of the container 11. The remaining second portion 9 of the entry port 1 is flexible and is defined by and extends through an open area in the seal or weld 8 from the rigid portion 10 to the interior space 13 of the container 11. In combination, the first portion 10 and the second portion 9 of inlet port 1 allow the passing of fluid into the main body portion 13 of the container 11. It is understood with respect to the inlet port 1 that many variations, including, but not limited to, making the port 1 entirely flexible, may exist that still fall within the spirit and scope of the invention. It is preferable, however, that at least a flexible portion 9 be included to facilitate sealing of port 1 closed as described hereinbelow.

Exit port 3 is preferably similar to port 1 at least in that it extends preferably from the exterior of the container 11 to the interior space 13 of the container 11 to allow fluid to flow out of the container 11. However, in order to provide selective fluid communication between the interior 13 of the container 11 and the exterior of the container, a frangible mechanism or connector 12 is preferably used inside exit port 3. Numerous such frangible connection mechanisms are known in the art. Rupturing the frangible mechanism 12 allows a portion of the mechanism (not specifically shown) to be separated from another portion (not specifically shown) of the frangible mechanism thereby permitting fluid to flow through port 3 out of the container 11. Examples of frangible mechanisms that could be used in this invention are described in detail in U.S. Pat. Nos. 4,340,049 to Munsch and 5,330,464 to Mathias, although it is understood that other well known frangible connectors could alternatively be used. Although in the described embodiment a frangible mechanism 12 is used, any means for maintaining sterility of the container as well as allowing selective access between the interior of the container and the exterior of the container may be used. The frangible connector 12 need only prevent fluid from flowing into the port 3 until the fluid is to be emptied from the container 11.

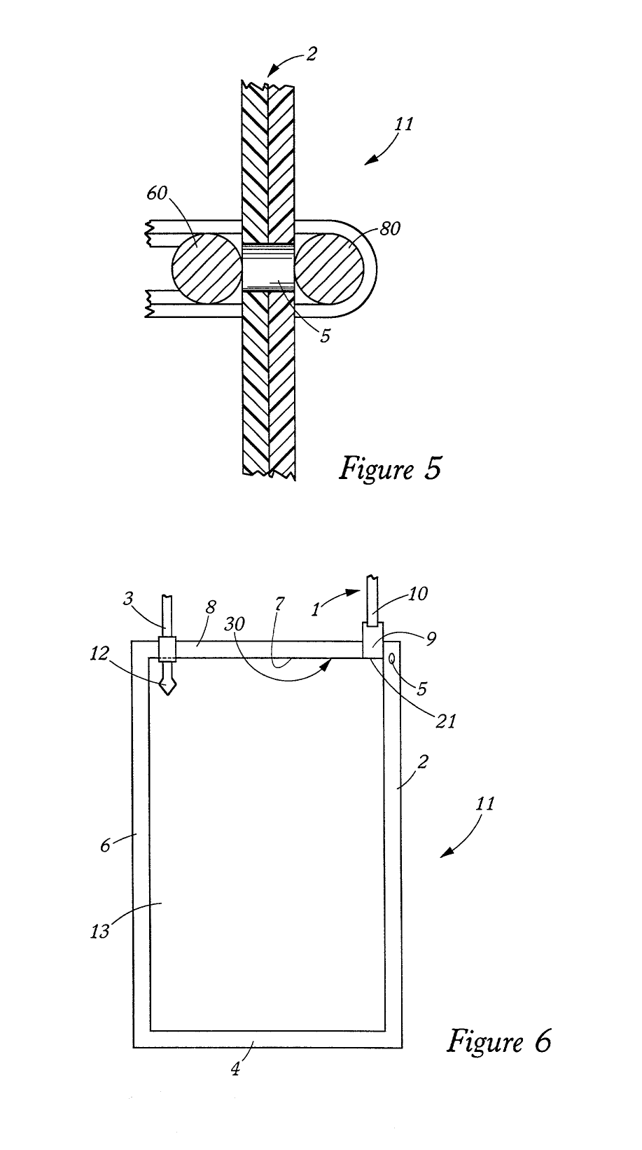

A hole 5 is preferably defined within the pre-formed manufactured seal 2 in the outer border zone of the container 11 and preferably is aligned with the interior edge 7 of the pre-formed manufactured seal 8. The hole 5 in the pre-formed seal 2 of the container 11 serves as an alignment indicator to indicate where the jaws of a welder or sealer commonly used to create seals or welds (see FIG. 5) will preferably be placed. Though preferred, the hole 5 need not be defined in the pre-formed manufactured seal 2, but may otherwise be defined by the cooperation of the welder/sealer and the location on the container 11 of the port 1 or 3 to be sealed. Further, the alignment indicator need not be a hole 5, but could be a mark or other type of feature of any kind including, for example, a notch in the pre-formed manufactured seal, or a type of weld (not shown in FIG. 1), so long as it preferably visually directs the operator of the welder or sealer to the location necessary to enable the creation of a continuous seal. Furthermore, the alignment indicator 5 preferably is, but need not be, aligned in all embodiments with the interior edge 7 of the pre-formed manufactured seal 2. The alignment indicator need only provide sufficient indication to the operator of the sealer of the desired sealing location preferable in the flexible portion 9 of port 1 to form a continuous seal 21 (see FIG. 6) across the port 1 to seal the container 11 closed. By the term continuous seal is meant a seal that closes the inlet port 1 and which approximately aligns with the lower inside edge 7 of the pre-formed manufactured seal 8, to create an uninterrupted seal across the width of the container and to isolate the port 1 from the main body portion 13 of the container 11.

If the container 11 does not have seals or welds along the outer edges of the container, other alignment methods could be used, including, but not limited to, a line or other marking for locating the proper sealing location for the sealer.

The hole or other type of guiding feature 5 may also serve to mechanically guide the sealer to the exact location on the container to be sealed. If a hand-held sealer is used, one jaw or other physical portion (not shown) of the sealer may be inserted through the opening of the hole 5 to mechanically direct the sealer to the exact location necessary to make a seal 21 continuous with the pre-formed factory seal 8.

One hand-held sealer which may be used with this invention as described in detail below, is a hand-held radio frequency (RF) welder such as the type manufactured by Sebra Company, Tucson, Ariz. However, use of any type of electromagnetic or heat sealer by any known manufacturer is contemplated to be within the scope of this invention. It is also contemplated that the electromagnetic or heat sealer can be stationary rather than hand-held such as those sealers used in known container sealing and manufacturing processes.

FIG. 2 shows a cross-sectional view of the container 11 and jaws 60 and 80 of a hand-held sealer 19 being placed over the location on the polymeric container 11 which is to be sealed. The two jaws of sealer 19 are a lower jaw 60 and an upper jaw 80. The upper jaw 80 and the lower jaw 60 of the sealer 19 are positioned on opposite sides of the container 11 over and under the alignment hole 5 (see FIGS. 1 and 5) to direct the operator to the specific location preferably of flexible portion 9 to seal off the entry port 1 while creating a continuous seal 21 (FIG. 6) which aligns approximately with the interior edge 7 of the pre-formed seal edge 8 (FIGS. 1 and 6).

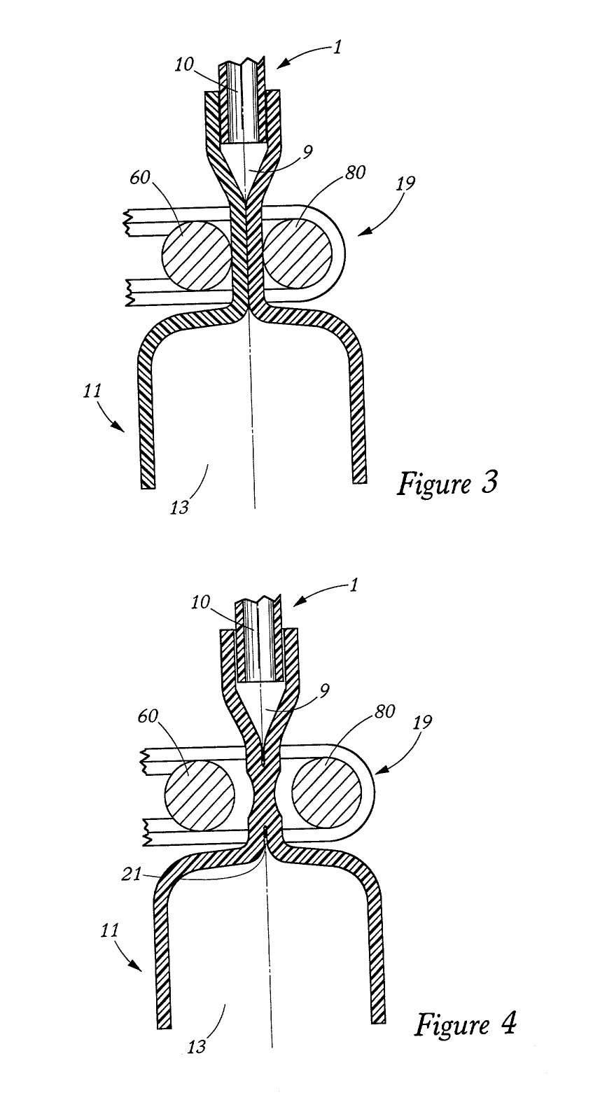

FIG. 3 is a cross-sectional view of the container 11 and both jaws 60 and 80 of the heat sealer 19 closed to seal off the entry port 1 from the main body portion 13 of the container 11. The flexible portion 9 of the port 1 is placed between the upper jaw 60 and lower jaw 80 of the sealer 19. The upper 60 and lower 80 jaws in a preferred embodiment are moved towards each other by a mechanism (not shown) resident on the heat sealer 19 until they come into contact with the exterior surface of the flexible portion 9 of the port 1. The jaws 60 and 80, again, in one known embodiment of a sealer 19, compress the flexible portion 9 of the port 1 until the port 1 is squeezed together, interrupting fluid communication between the entry port 1 and the interior of the container 13. In one preferred embodiment where a radio frequency welder is used, such as the Sebra® sealer noted above, RF energy is applied to and between the upper jaw 60 and lower jaw 80, respectively, to create an electromagnetic energy field between the jaws. The electromagnetic energy field causes dielectric heating and resultant melting of the flexible portion of the port 9 to create a welded seal thereacross. When jaws 60 and 80 respectively of the sealer 19 are released from the container after the application of RF energy a seal 21 (FIGS. 4 and 6) will have been formed. As mentioned above, the RF sealer does not have to be a hand held sealer such as that described above. The RF sealer may be a bench top or other type of stationary sealer as is known in the art, without departing from the spirit and scope of the invention. It should also be noted that a heat sealer may be used to form isolating seal 21. The seal 21 isolates the port 1 from the main body portion 13 of the container 11 and prevents fluid communication between the entry port 1 and the main body portion 13 of the container 11.

FIG. 4 shows a cross-sectional view of the container 11 and open jaws 60 and 80 of the sealer 19 after the sealing process has been accomplished. Jaws 60 and 80 respectively of the sealer 19 are released from the bag after the application of RF energy. Seal 21 is formed after RF energy has been applied by the jaws 60 and 80 as described above. Seal 21 isolates the port 1 from the main body portion 13 of the bag 11 and prevents fluid communication between the entry port 1 and main body portion 13 of the bag 11.

FIG. 5 shows a cross-sectional view of the preferred container 11 with the open jaws 60 and 80 of the sealer 19 aligned over the alignment hole 5 in preparation for sealing the entry port 1 closed. As described above, alignment hole 5 is located in the pre-formed manufacturing seal 2 (see FIG. 1) of the container 11. The alignment hole 5 is placed between the upper jaw 60 and lower jaw 80 of the sealer 19 to direct the operator of the sealer to the location preferred to create a continuous seal. Alternatively (not shown) one jaw of the RF welder may actually be passed through the hole 5 to align with the pre-formed manufactured seal 2.

FIG. 6 shows a top view of the container 11 after the entry port 1 has been sealed off from the main body portion 13 of the container 11. Seal 21 creates a new continuous seal 30 across the length of the container, approximately aligned with the lower edge 7 of the pre-formed manufactured seal 8. The flexible portion 9 of the entry port 1 has thus been sealed closed using the sealer 19 (as shown in FIGS. 2-5). The entry port 1 is then fluidly separated from the main body portion 13 of the container 11. In the preferred embodiment of this invention, it is the entry port 1 which is sealed off from the main body portion 13 of the container 11. However, any other port located anywhere on the container, such as port 3, or other ports (not shown) could be sealed off in a manner according to the present invention.

One preferred method of operation using the container 11 as described above is as follows. Initially, fluid which is to be pathogen inactivated flows into the container 11 via the entry port 1. The fluid may be mixed with a photosensitizer before being transferred into the container, or the fluid and the photosensitizing agent could be mixed within the container itself. The photosensitizer could be in the container 11 before entry of the fluid or added through a port (to be sealed similar to the manner of port 1). The entry port 1 is then, prior to the inactivation procedure, sealed off from the main body portion of the container 13 with a seal which is substantially continuous with the lower edge 7 of the pre-formed manufactured seal 8. Sealing the entry port 1 off from the main body portion 13 of the container 11 through use of a continuous seal is desirable, particularly in viral or photosensitizing methods as it separates or isolates the port 1 from the main body portion 13. Without a continuous seal such as described herein, fluid may become trapped in undesirable areas such as within port 1, creating stagnant pockets of fluid. The presence of stagnant pockets of fluid trapped inside the container 11 may prevent the photosensitizing agent from coming into inactivating interaction with any pathogens contained within the stagnant fluid pockets. Also, if irradiation is used, the relatively rigid port 10 may block proper irradiation of any fluid contained therein.

While the fluid inside the main body portion 13 of the container or bag 11 is being inactivated, the exit port 3 is also isolated from the main body portion 13 of the container 11 by the frangible mechanism 12. The frangible mechanism allows the fluid in the interior or main body portion 13 of the container 11 to flow out of the container only after the frangible mechanism 12 is broken, as is known and/or described above. Once the fluid has undergone the process of photosensitizing, the operator can rupture the frangible mechanism 12 by manipulatively bending a portion of the mechanism 12 according to its method of use to break open the port 3 and thereby allow the fluid to flow out of the container 11 through the exit port 3.

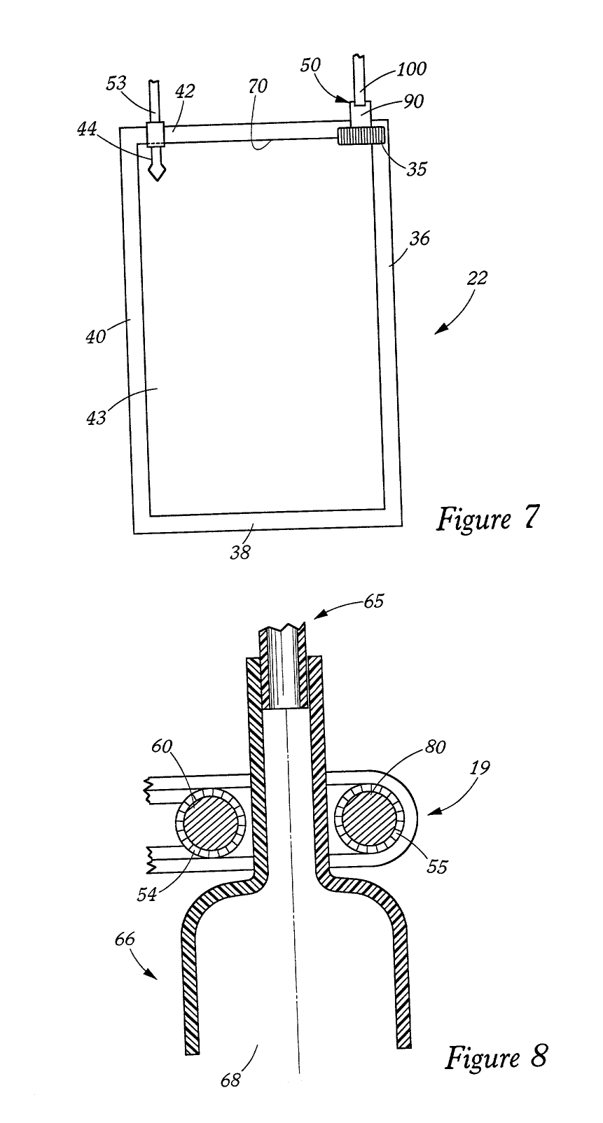

In the above described embodiment, the polymeric container or bag 11 may preferably be made of an RF excitable material such as PVC. However, a material other than PVC may be used in the manufacture of the container 11. In another embodiment, a polyolefin polymeric material (e.g. polyethylene or polypropylene) may be used to manufacture the container 11. However, polyolefins are not as excitable in response to RF energy, and thus are less able to be sealed using a standard RF sealer. To form seals in the polyolefin material, it may be necessary to place a layer of RF excitable material such as PVC, over the location where the seal is to be formed. An exemplary embodiment is depicted in FIG. 7 which shows another embodiment of the present invention including an alternative polyolefin or other substantially non-RF excitable container 22. In a manner similar to that described above in FIG. 1, the container 22 is sealed or welded during manufacture to form pre-formed seals or welds 36, 38, 40, and 42 respectively, in the outer border zones around all sides of the container except for an entry port 50 and an exit port 53. The continuous pre-formed seals or welds create an interior space 43 therebetween. The two ports or openings 53 and 50 allow fluid ingress and egress; that is, an entry port 50 provides fluid ingress and an exit port 53 provides fluid egress. In FIG. 7, the container 22 is shown with entry port 50 open to allow the fluid access to the interior 43 of the container 22. Interior 43 is also interchangeably called the main body portion of the container 22. As shown in FIG. 7, the container 22 contains two ports; however, as above, the container 22 may also contain any number of ports and still remain within the spirit and scope of the present invention.

Entry port 50, similar to port 1 in the first embodiment, preferably has two portions, a relatively rigid first portion 100 and a flexible second portion 90. The relatively rigid first portion 100 extends from the exterior of the container toward the interior, or main body portion 43 of the container 22. The remaining second portion 90 of the entry port 50 is flexible, and is defined by and extends through an opening in the seal or weld 42 from the partially extended rigid portion 100 to the interior of the container 43. In combination, the first portion 100 and the second portion 90 provide fluid communication to and thereby allow fluid to flow into the main body portion 43 of the container 22. It is understood with respect to the port 50 that many variations may exist that still fall within the spirit and scope of the invention. For example, as with the first embodiment described herein above, the port 50 may be entirely flexible.

Exit port 53 also preferably extends from the exterior of the container to the interior 43 of the container 22 to allow fluid to flow out of the container 22. In order to provide selective fluid communication between the interior 43 of the container 22 and the exterior of the container through exit port 53, a frangible mechanism 44 is preferably used inside exit port 53. Rupturing the frangible mechanism 44 as is known in the art allows a portion of the mechanism (not specifically shown) to be separated from another portion of the mechanism (not specifically shown) thereby permitting fluid to flow out of the container 22 through exit port 53. Until the frangible mechanism 44 is ruptured, exit port 53 is separated and isolated from main body portion 43.

In the embodiment of FIGS. 1-6, a hole 5 was described as the indicator which served to align the application of a sealer. In FIG. 7, instead of the alignment hole 5 a layer of PVC material or tape 35 may be located over the inlet port 50. The layer of material or tape 35 may then serve as a guide to the location where the jaws of a sealer or welder commonly used to create seals or welds (see FIG. 5) is preferably to be placed. The tape 35 can also extend over the pre-formed manufactured seals 36 and 42 as shown. The tape 35 is preferably made out of electromagnetic and more preferably RF excitable material of which PVC is one example. When exposed to RF energy, the layer of excitable material melts the polyolefin material underneath, creating a seal that separates and/or isolates the inlet port 50 from the main body portion 43. It is understood that similar RF excitable material or tape can be used at other locations on containers of non-RF excitable material to bring about sealing of such containers.

FIG. 8 illustrates another alternative embodiment of the present invention that is similar to the embodiments of FIGS. 1-6 except that the container 66 (similar to container 11, see FIG. 1) here is made from material that is not as RF excitable as the PVC used in container 11. A preferred material here, as in FIG. 7, is a polyolefin. FIG. 8 shows a cross-sectional view of the distinctive jaws 60 and 80 of a hand-held sealer 19 being placed over the location on the polyolefin container 66 which is to be sealed. In this embodiment, the alignment indicator is not the RF excitable tape (as was described in the embodiment of FIG. 7). Rather, the alignment indicator is again, preferably, a hole or other type of guiding feature (not shown) in the pre-formed manufactured seal (not shown) similar to that shown in and described relative to FIG. 1 above. The sealer 19, as in the previous embodiments, has two jaws, a lower jaw 60 and an upper jaw 80. However, in this embodiment, a lower RF excitable sleeve 54 is selectively engaged on the lower jaw 60 and an upper RF excitable sleeve 55 is selectively engaged with the upper jaw 80 of the sealer 19. The lower jaw 60 and its lower RF excitable sleeve 54 and the upper jaw 80 and its upper RF excitable sleeve 55 of the sealer 19 are positioned on opposite sides of the container 66 over and under an alignment indicator (not shown) similar to hole 5 (see FIGS. 1 and 5) to direct the operator to the specific location necessary to seal off the entry port 65 while creating a continuous seal which aligns with the lower edge (not shown) of the pre-formed seal (not shown, see, e.g., 42 of FIG. 7). When the lower RF excitable sleeve 54 and the upper RF excitable sleeve 55 are selectively engaged on the lower jaw 60 and upper jaw 80 of the sealer 19, and exposed to RF energy, the sleeves 54 and 55 melt the polyolefin material of the container 66 so that the sealer may directly seal the entry port 65 off from the main body portion 68 of the container 66 in a continuous seal without the use of PVC tape applied to the container itself (as shown in the embodiment of FIG. 7 above). Alternatively, heat could be used to seal the polyolefin material, without the need for RF excitable elements.

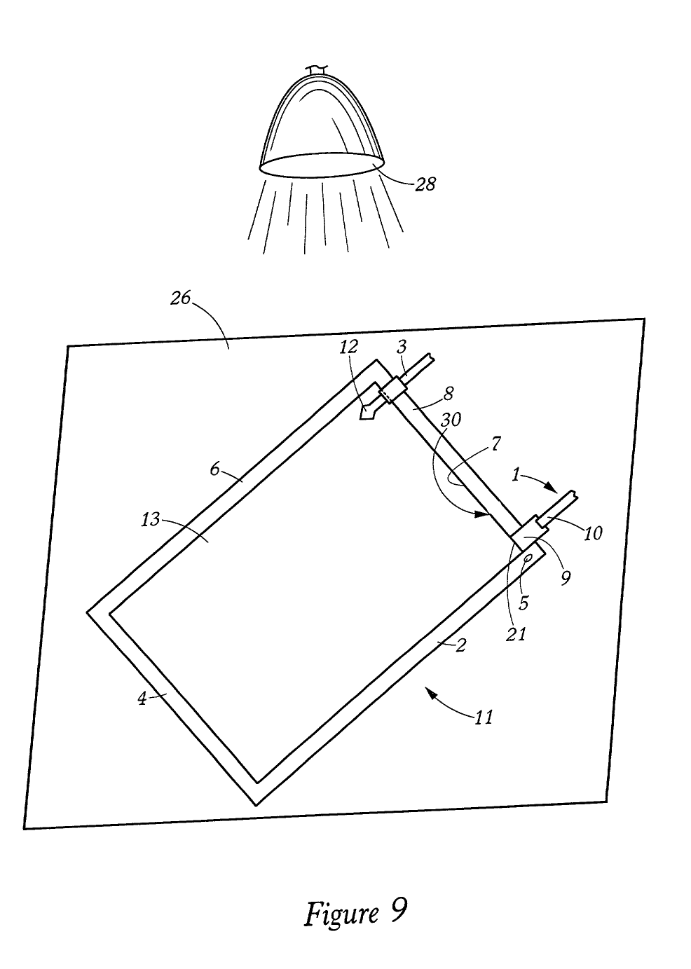

The use of any of the alternative containers 11, 22 and 66 will now be described with respect to FIG. 9. Although the description is here directed specifically at the container embodiment of FIGS. 1-6, the process described can also be used with the containers described in the alternative embodiments. FIG. 9 depicts an embodiment of this invention in which the fluid to be decontaminated flows into the blood container 11 through an inlet or entry port 1. The inlet port is then sealed using a sealer to create a continuous seal as described above. A photosensizing agent, preferably a photosensitizer (not shown) may be prepackaged in the container 11, or the pathogen inactivation photosensitizer may be added to the fluid before or after the fluid to be irradiated flows into the container 11. A shaker table 26 is preferably used also to agitate the container 11 to homogeneously mix the photosensitizer with the fluid to be irradiated and is activated, while a photoradiation source 28 is activated to irradiate the fluid containing the photosensitizer in the container 11. Light of an appropriate wavelength is preferably used to actuate the photosensitizer to inactivate any pathogens or viruses contained within the fluid. The outlet port 3 remains isolated from the main body portion 13 of the container 11 during this irradiation and photosensitizing process. The decontaminated fluid may then be stored in container 11 until it is ready to be transfused. Alternatively, the decontaminated fluid may be transferred to another storage container. When it is desirable to empty container 11, frangible mechanism 12 is broken and fluid is passed through outlet port 3.

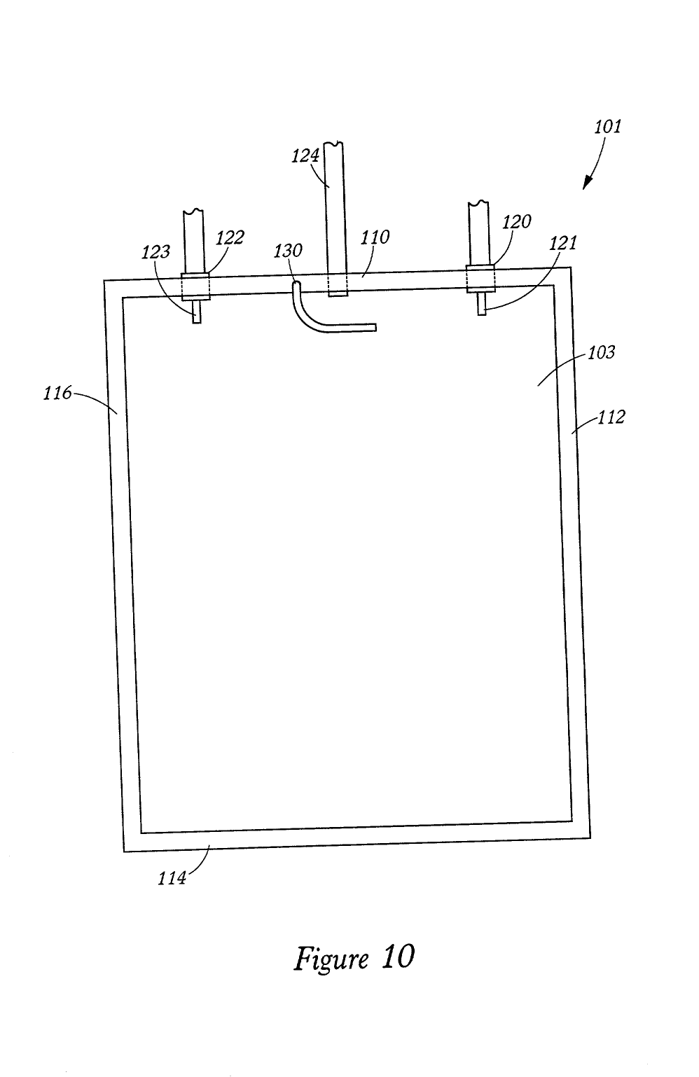

FIG. 10 is a top plan view of another embodiment of a photopermeable container for use in inactivating any pathogens contained in the fluid. The container or bag 101 is preferably made of sheets of a polymeric type material which is sealed or welded around its outer border zones during manufacture in a continuous manner to form pre-formed seals or welds 110, 112, 114 and 116 respectively, around all sides of the container 101. Continuous here refers to the hermetic fluid-tight seal around the entire periphery of bag 101. The continuous pre-formed seals or welds create an interior space or main body portion 103. It should be noted that container 101 may be formed in such a manner so as to not have seals around all sides of the container, and still comply with the spirit and scope of the invention. As shown in FIG. 10, container 101 has two substantially conventional ports 120, 122 or openings extending through the pre-formed seals or welds 110, 112, 114 and 116. Port 120 provides for fluid ingress and/or fluid egress and port 122 also provides for fluid ingress and/or fluid egress into and out of the container 101. In the preferred embodiment described in FIG. 10, the container 101 is shown having two substantially conventional ports, and a third port which will be described in more detail below; however, container 101 could have any number of ports as required for its particular purpose and still remain within the spirit and scope of the present invention. Closed internal frangible connector ends 121 and 123 respectively, extend from each conventional port 120 and 122 into the interior 103 of the container 101. As shown in FIG. 10, the frangible connector ends 121 and 123 are intact, and serve to close off the ports in a fluid tight manner. Frangible connector ends 121 and 123 may be broken away from ports 120 and 122 by application of finger pressure, which breaks connector ends 121 and 123 off, allowing for fluid flow into and/or out of container 101. Numerous such frangible connection mechanisms are known in the art. Examples of frangible mechanisms that could be used in this invention are described in detail in U.S. Pat. Nos. 4,340,049 to Munsch and 5,330,464 to Mathias, although it is understood that other well known frangible connectors could alternatively be used. Although in the described embodiment breakaway frangible mechanisms are used, any means for maintaining sterility of the container as well as for allowing selective access between the interior of the container and the exterior of the container may be used. An alternative embodiment may be the type of frangible connector described with reference to FIG. 1 above. The frangible connectors 121 and 123 need only prevent fluid from flowing into or out of ports 120 or 122 until such time as the fluid is to enter or be emptied from the container 101. In the preferred embodiment, ports 120 and 122 are egress ports, used for emptying the bag. Although not shown, a bulb for removing samples for testing, and/or a filter for filtering out unwanted substances from the fluid contained within the bag may be fluidly attached to either egress port 120 or 122.

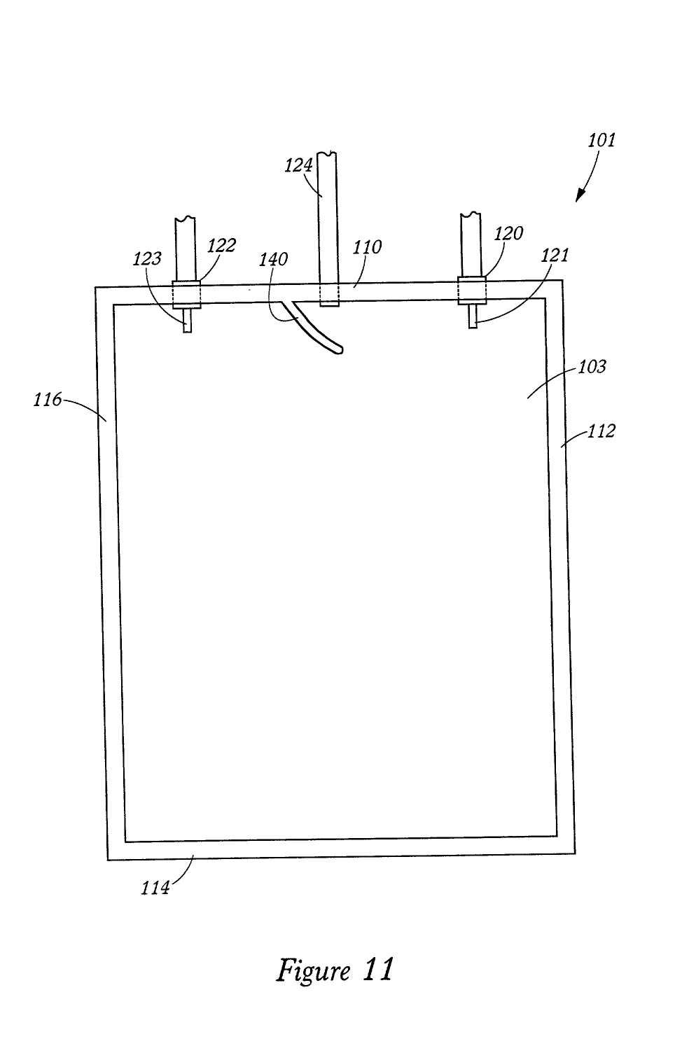

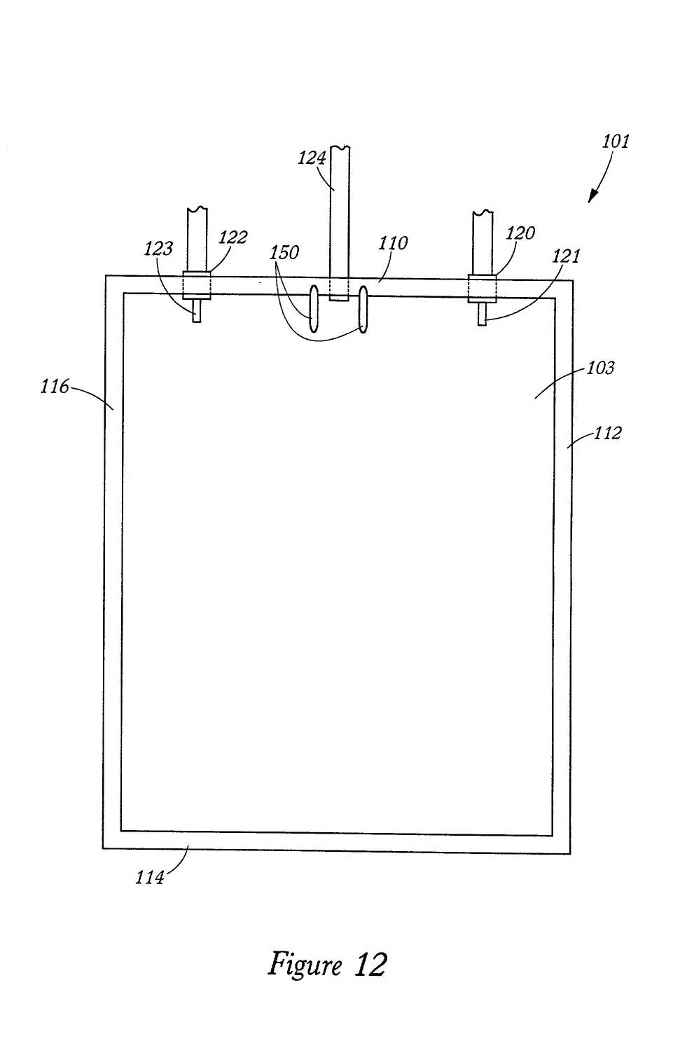

In between substantially conventional ports 120 and 122 is a third port, which is preferably a flexible tube 124 which may extend into the interior 103 of bag 101 through pre-formed seal or weld 110. It is envisioned that tube 124 maintains its flexibility in its entirety as it extends through seal 110. However, a substantially conventional rigid or semi-rigid port similar to ports 120 and 122 may also be used without departing from the spirit and scope of the invention. Flexible tube 124 preferably extends just past seal 110 into the interior 103 of the container 101. In an alternative embodiment (not shown) the flexible tube 124 extends through seal 110 and terminates at the end of seal 110. In this embodiment, the flexible tube 124 does not extend into the interior of the container, although the opening of flexible tube 124 opens into the interior 103 of the container 101. Tube 124 is preferably made of a PVC type material. An internal seal or weld 130 is located in close proximity to the end of tube 124, extending into the interior of the bag 103. A preferably “L-shape” weld 130 extends downward from seal 110 and curves past the end of tube 124 towards and past the opposite side of tube 124, to partially surround the end of the flexible tube on three sides leaving one open side. It is understood that weld 130 may be pre-manufactured into the bag, or may be added prior to filling the bag with fluid. Weld 130 may be contiguous with upper seal 110, or may extend into seal 110, in a manner such as that shown in FIG. 10. Other shapes of welds 130 may be used as well, without departing from the spirit and scope of the invention. As shown in FIG. 11, a “back slash” shape or diagonal weld 140 may also be used. This weld 140 is shown contiguous with upper seal 110. Weld 140 may also extend into seal 110 in a manner similar to that shown in FIG. 10. In another embodiment shown in FIG. 12, flexible tubing 124 may be surrounded by two parallel “I-shape” seals or welds 150 which extend from seal 110 into the interior of bag 103 to a point below the end of tubing 124. Although not shown, a “J-shape” weld is another alternative which may be used in the above described invention. It should be noted that although the alternative embodiments set forth in FIGS. 10, 11 and 12 differ in the shape of the pre-manufactured welds 130, 140, 150 used to seal tubing 124 off from the main body portion of the bag 103, the elements which are common to all embodiments have been given all the same numerals.

In the preferred embodiment, it is envisioned that flexible tubing 124 is used for fluid egress into the bag.

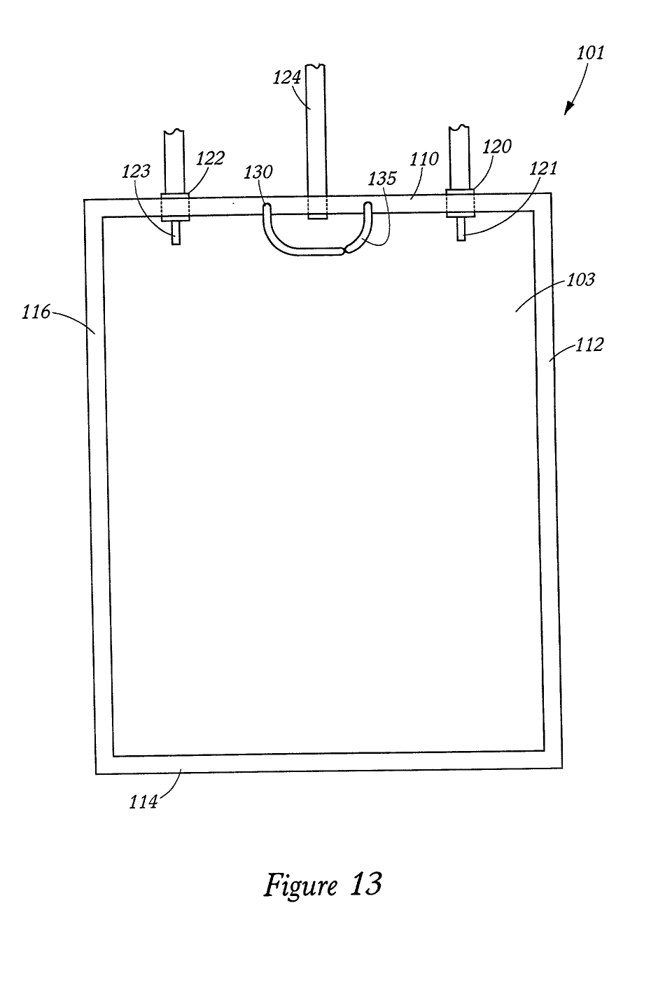

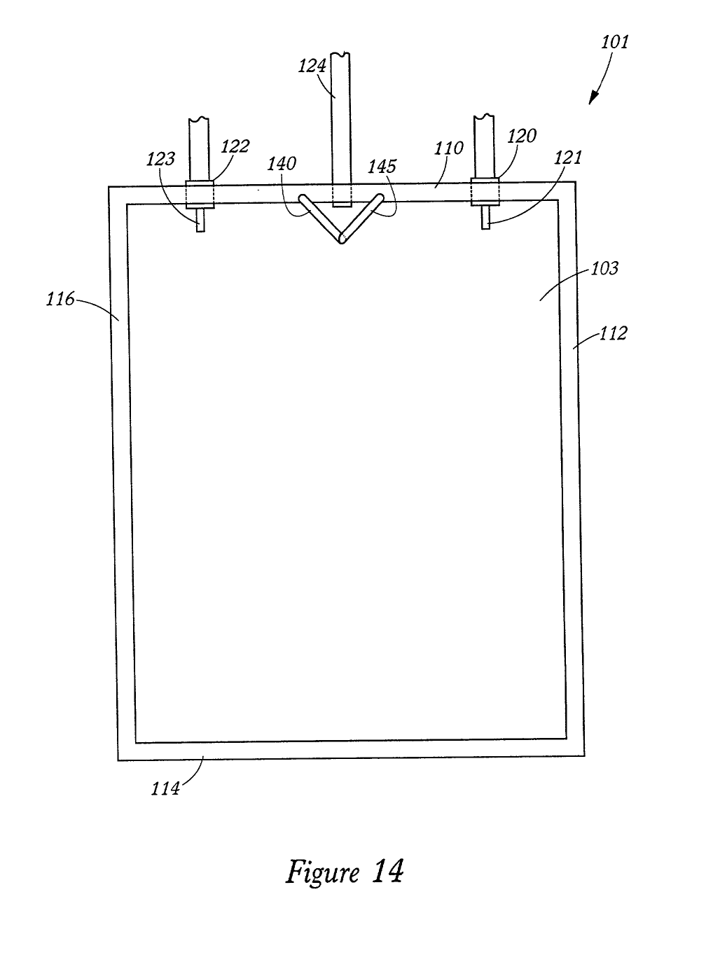

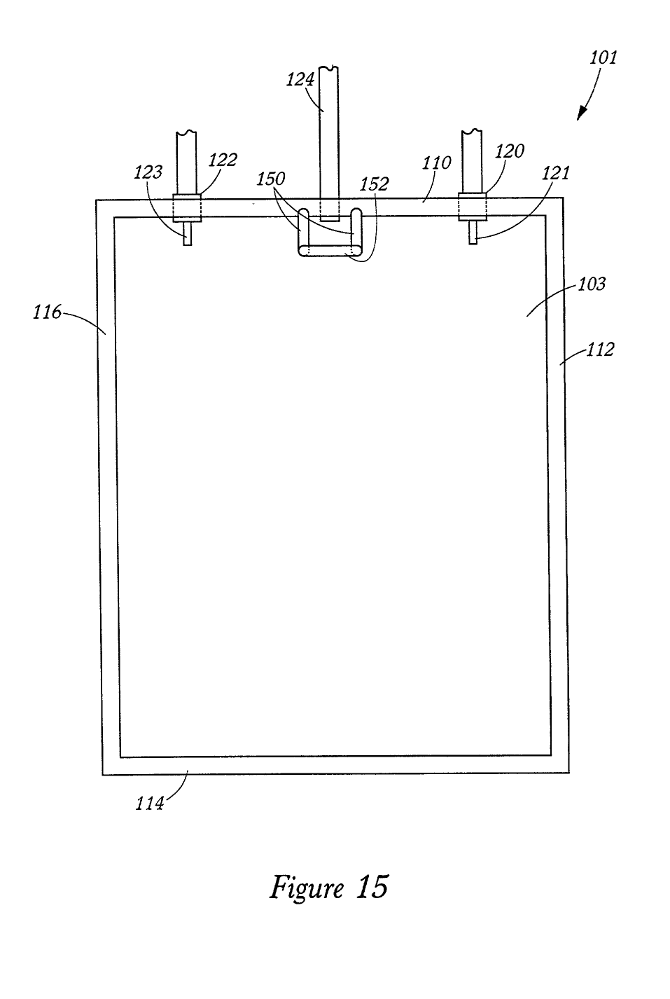

One method of using the alternative embodiment of FIGS. 10, 11 and 12 are as follows. Initially, fluid which is to be pathogen inactivated may flow into container 101 via tube 124. The fluid may be mixed with a photosensitizer before being transferred into the container, or the fluid and the photosensitizing agent could be mixed within the container itself. The photosensitizer could be in the container 101 before entry of the fluid or added through a port. The fluid flows along the “L-shape” 130 (see FIG. 10) or “back slash” shape 140 (see FIG. 11) or through the parallel “I-shape” (see FIG. 12) welds and into bag 101. After fluid has entered the bag, tube 124 is sealed off from the interior 103 of the bag 101. This is done by using a RF sealer or a heat sealer to seal the open side, extending the weld to make a seal which surrounds the end of the tube 124 on all four sides, separating the tube 124 from the interior 103 of the container 101. Use of the hand held sealer is described with reference to FIG. 2 above. As shown in FIG. 13, the newly formed seal 135 extends from the end of the “L-shape” weld 130 to the seal 110. The newly created seal 135 may be created in a contiguous manner with the initial “L-shape” pre-formed seal 130, or may overlap both the end of the “L-shape” seal 130 as well as upper seal 110. Either way, the effect is to seal tubing 124 off from the interior of bag 103 by the newly created “U-shape” seal or weld 135. FIG. 14 shows the “back slash” shape weld 140 newly welded into a substantially “V-shape” seal 145, which also separates tubing 124 from the main body portion of the bag 103 in substantially the same manner as that described above with reference to FIG. 13. As shown in FIG. 15, a horizontal weld 152 may be used to connect the parallel “I-shape” welds to seal off the interior of bag 103 to form another substantially “U-shape” seal. Connection of the “I-shape” welds could be done by using a modified head design for a RF hand held sealer, or could be done by using a non-hand held sealer, such as one that is placed on a bench or a table top.

Sealing tube 124, off from the main body portion 103 of the container 101 through use of a continuous seal is desirable, particularly in pathogen inactivating or photosensitizing methods. Without a continuous seal such as described herein, fluid may become trapped in undesirable areas such as within the tubing 124 or ports 120 and/or 122, creating stagnant pockets of fluid. The presence of stagnant pockets of fluid may prevent the photosensitizing agent from coming into inactivating interaction with any pathogens contained within the stagnant fluid pockets. Also, if irradiation is used, the rigid portion of ports 120 and 122 may block proper irradiation of any fluid contained therein.

Virtually any method known in the art may be used for making the polymeric bags shown in the alternative embodiments of FIGS. 10 through 15. The polymeric bags may be blow molded, made out of a single piece of flexible polymeric material folded in half and welded around three sides, or made out of two pieces of flexible polymeric material welded together around all four sides. The port or ports 120 and 122, as well as the flexible tube 124 may be extended into the main body portion of bag 103 before, during, or after the initial welding process. The internal seal 130, 140 or 150 which partially surrounds the end of the flexible tubing 124 may be sealed or welded in location using a sealer such as that shown in FIG. 2, after placement of the tubing 124. Alternatively, the seals or welds 130, 140, or 150 may be welded within bag 101 first, followed by port or tubing 124 placement within the partially surrounding seal 110. After fluid is introduced into the bag 101 via tubing 124, the open side of the seal or weld 130, 140 or 150 is sealed closed, to create a continuous seal around all four sides of the tubing 124, separating the tubing 124 from the main body portion 103 of the bag 101.

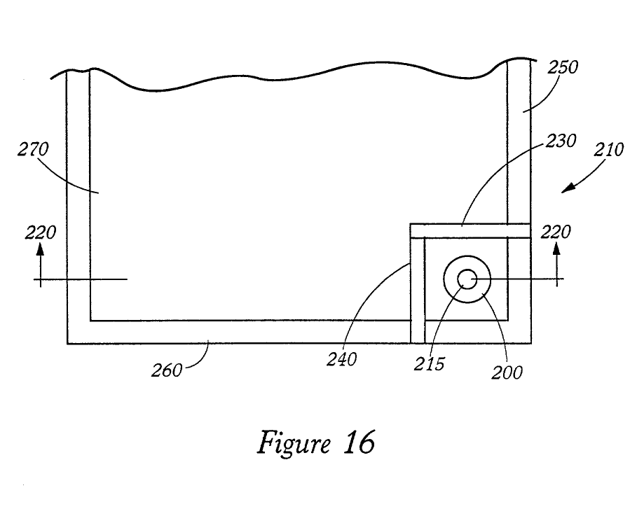

An alternative type of port may be used in addition to or in replacement of the ports described above. The alternative port is described in relation to FIG. 16. The port 200 is shown as extending from within the top or front portion 270 of bag 210 in an upward direction. However, the port 200 may extend from the bottom or back portion (not shown as a specific element) as well without departing from the spirit and scope of the invention. The port shown in FIG. 16 is an injection molded port 200. The port 200 is typically welded through a sheet of polymeric film, shown here as being the top or front portion of the bag 270. The port 200 extends through the film of the top or front portion 270 of the bag into the interior of the bag 210. A tube 215 for adding or removing fluid to and from the bag 210 is then bonded or welded to the interior portion of the port (not shown). Port 200 is shown as being located in a corner between two pre-formed seals 250 and 260 of the bag. Such a location enables the port to be easily sealed off from the main body portion of the bag by a sealer, such as the type described above. It should be noted however, that port 200 may be located anywhere within the front or back portion or any side, top or bottom of bag 210, and still be sealed off from the main body portion of the bag. Internal weld or seal 230 extends from the pre-manufactured seal 250 into the interior of the bag, past port 200. As shown, seal 230 is in a substantially “I-shape,” however, any other shape including any previously described may be used with this invention. Once fluid to be inactivated is flowed into the bag 210 via tube 215, port 200 may be sealed off from the body of the container using a sealer such as that previously described. The new seal or weld 240 formed by the sealer extends from pre-formed seal 260 to the substantially “I-shaped” seal 230, completely closing off port 200 from the interior body portion of the container. Seals 230 and 240 are shown as extending over pre-formed seals 250 and 260. It is understood however, that the only requirement is that the seals formed by the sealer abut the already sealed portions to isolate port 200 from the inner portion of the bag 210. It is not necessary that seals 230, 240, 250 and 260 overlap if not needed for the proper isolation of the port. It is further understood that other well known types of ports could also be used with the instant invention. The only requirement for a port is that it be capable of being isolated from the main body portion of the bag.

It should be understood that various changes and modifications to the presently preferred embodiments described herein will be apparent to those skilled in the art. It is foreseeable that the shape of the container, the number of ports located on the container, as well as the location of the ports on the container may be varied. It is also foreseeable that the container may be manufactured out of a plurality of different polymeric materials. The use of different types of sealers is contemplated as well. These examples are not meant to be limiting, but rather are exemplary of the modifications that can be made without departing from the spirit and scope of the present invention and without diminishing its attendant advantages.

What is claimed is:

1. A method for inactivating pathogens in a fluid which may contain pathogens; comprising the steps of:
flowing a fluid which may contain pathogens into a container having a main body portion and a first port, through the first port into the main body portion of the container; and
sealing the first port closed to create a continuous seal across the container to isolate the first port from the main body portion of the container.

2. The method of claim 1 further comprising adding a photosensitizer to the fluid to be inactivated.

3. The method of claim 2 further comprising exposing the fluid and photosensitizer to light of an appropriate wavelength.

4. The method of claim 1 wherein the container has a second port with a frangible mechanism and wherein the method further comprises:
breaking the frangible mechanism to open the second port in the container.

5. The method of claim 4 further comprising:
flowing the fluid from the main body portion through the second port in the container.

6. The method of claim 1 wherein the step of sealing the first port closed to create a continuous seal across the container comprises the step of sealing the first port with an electromagnetic sealer.

7. The method of claim 1 wherein the step of sealing the first port closed to create a continuous seal across the container comprises the step of sealing the first port with a heat sealer.

8. The method of claim 1 wherein the step of sealing the first port closed to create a continuous seal comprises:
aligning the container at a predetermined location between an upper jaw and a lower jaw of a sealer;
compressing the jaws of the sealer; and
applying energy to create a seal.

9. The method of claim 8 wherein the step of applying energy comprises applying radio frequency electromagnetic energy to radio frequency excitable material on the container.

10. The method of claim 1 wherein the step of sealing the first port closed to create a continuous seal comprises:
aligning the container at a predetermined location between the jaws of a sealer; and
compressing the jaws of the sealer.

11. The method of claim 1 wherein the step of sealing the first port closed to create a continuous seal comprises:
providing a radio frequency sealer having an upper jaw and a lower jaw;
placing a radio frequency excitable sleeve on the upper jaw;
placing a radio frequency excitable sleeve on the lower jaw,
aligning the container at a predetermined location between the upper jaw and the lower jaw;
compressing the jaws of the sealer together;
applying radio frequency electromagnetic energy to dielectrically heat the container to create a seal.

12. A container for containing fluid to be pathogen inactivated comprising
a main body portion to contain the fluid to be pathogen inactivated;
an entry port for passing fluid into the main body portion;
an alignment indicator on the container wherein said alignment indicator provides for aligning a sealer to a sealing location to seal the container and isolate the entry port from the main body portion.

13. The sealer of claim 12 wherein the sealer is a hand-held sealer.

14. The sealer of claim 12 wherein the sealer is a heat sealer.

15. The container of claim 12 wherein the container is photopermeable.

16. The container of claim 12 where the entry port comprises a relatively rigid first portion and a flexible second portion and wherein the alignment indicator provides for aligning a sealer to seal the flexible second portion of the entry port.

17. The container of claim 12 wherein the container comprises an exit port.

18. The container of claim 17 wherein the exit port includes a frangible connector.

19. The container of claim 12 further comprising a first weld on one side of the container wherein said alignment indicator comprises a hole in said first weld.

20. The container as claimed of claim 12 wherein said alignment indicator comprises a mark on the container.

21. The container as of claim 12 wherein the container comprises PVC material.

22. The container as claimed of claim 12 wherein the container comprises a polyolefin material.

23. The container of claim 12 wherein the alignment indicator comprises an RF excitable material.

24. The container of claim 23 wherein said RF excitable material is PVC.

25. The container of claim 23 wherein said RF excitable material is PVC tape.

26. The container of claim 23 further comprising a pre-formed manufactured seal having an interior edge and wherein said RF excitable material aligns with said interior edge of the pre-formed manufactured seal on the container.

27. The container of claim 12 further comprising
a pre-formed manufactured seal having an interior edge on at least one outer border zone of the container;
wherein the sealing location aligns with an interior edge of the pre-formed manufactured seal to isolate the inlet port from the main body portion of the container.

28. A container of non-electromagnetically excitable material for containing fluid to be irradiated comprising;
a main body portion to contain the fluid to be irradiated;
an entry port for passing fluid into the main body portion;
electromagnetically excitable sealing material on the container wherein the electromagnetically excitable material heats the container to create a seal when exposed to electromagnetic radiation.

29. The container of claim 28 wherein the container is photopermeable.

30. The container of claim 28 wherein the entry port comprises a relatively rigid first portion and a flexible second portion.

31. The container of claim 28 wherein the sealing material enables the use of a sealer to seal the container.

32. The container of claim 28 wherein the container comprises an exit port.

33. The container of claim 32 wherein the exit port comprises a frangible connector.

34. The container of claim 28 wherein the electromagnetically excitable material is PVC.

35. A container having at least one seal for containing fluid to be pathogen inactivated comprising:
a main body portion to contain fluid to be pathogen inactivated;
a flexible tube disposed within and through a seal to allow for fluid ingress into the main body portion of the container; and
a weld extending from the at least one seal into the container to partially surround the flexible tube to enable the tube to be sealed off from the main body portion by a sealer.

36. The container of claim 35 wherein the weld extends into the at least one seal.

37. The container of claim 35 wherein the container further contains at least one substantially conventional port.

38. A method for inactivating pathogens in a fluid which may contain pathogens; comprising the steps of:
flowing a fluid which may contain pathogens into a container having:
a main body portion;
at least one port disposed within and through a seal for flowing fluid into the main body of the container; and
a weld extending from the seal into the container to partially surround the port;
forming a seal which extends from the weld to separate the port from the main body portion of the container.

39. A container having one or more seals for containing fluid to be pathogen inactivated comprising:
a main body portion;
at least one port which extends into the main body portion; and
an internal weld in the main body portion which partially surrounds the port.

40. A container having one or more seals for containing fluid to be pathogen inactivated comprising:
a main body portion;
at least one port which extends through the one or more seals and terminates at the end of the one or more seals and
an internal weld in the main body portion which partially surrounds the port.

41. A container for containing fluid to be pathogen inactivated made from at least one sheet of flexible polymeric material and having at least one seal comprising:
a main body portion to contain the fluid to be pathogen inactivated;
at least one port extending from within the flexible polymeric material; and
a weld extending from the at least one seal into the container to partially surround the port to enable the port to be sealed off from the main body portion by a sealer.

42. The container of claim 41 wherein the port is injection molded.

43. A method of manufacturing a bag used for inactivating pathogens comprising the steps of:
providing a main body portion of a bag;
extending at least one port into the main body portion of the bag; and
internally sealing around the port to surround the port on three sides.
